SEQUENCE LISTING (17 sequences)

SEQ ID NO: 1 - protein, 891 aa, Escherichia coli
  REGION 1..891: note = MISC_FEATURE - E. coli bifunctional NAD+ dependent acetylating acetaldehyd/alcohol dehydrogenase (E. coli adhE) amino acid sequence
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 2 - protein, 467 aa, Escherichia coli
  REGION 1..467: note = MISC_FEATURE - E. coli ethanolamine utilizing protein (Ec_eutE) amino acid sequence
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 3 - protein, 455 aa, Lactobacillus plantarum
  REGION 1..455: note = MISC_FEATURE - L. plantarum acetaldehyde dehydrogenase (Lpla_acdH) amino acid sequence
MLKEMEETTVSRSIDRLVLNASLAANRLEVMDQSQVDQAVAAMARAAHAARGMLAAMAVEETGRGNYRDKVAKNDFAAKNVYNYIKDDKTVGIINDDPVSGVMKVAEPVGIIAGVTPVTNPTSTVIFNAMLALKTRNPIIFGFHPFAQKSCVETGRIIRDAAIASGAPKDWIQWIKTPSLEATNTLMNHPGVATIIATGGAGMVKTAYSTGKPALGVGPGNVPCFIEQTADIQQAVSDVVTSKSFDNGMICASESNLIVADQIYDQVKRELSHNGVYFVGTENFKALEATVMNLDKQAVDPKVAGQTPWQIAQWAGFDVPSDTKVLAVELPSIGGDQVLSREKLSPVLAVVHAKDTEAGFNLMKRSLALGGLGHTAALHTTDEAVMNKFALEMTACRALINVPSSQGAIGYKYDNVAPSLTLGCGTWGHNSISHNLEDWDLLNIKTVAKRLTKIR

SEQ ID NO: 4 - protein, 469 aa, Listeria innocua
  REGION 1..469: note = MISC_FEATURE - L. innocua acetaldehyde dehydrogenase (Linn_acdH) amino acid sequence
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 5 - protein, 869 aa, Staphylococcus aureus
  REGION 1..869: note = MISC_FEATURE - S. aureus acetaldehyde/alcohol dehydrogenase (Saur_adhE) amino acid sequence
MLTIPEKENRGSKEQEVAIMIDALADKGKKALEALSKKSQEEIDHIVHQMSLAAVDQHMVLAKLAHEETGRGIYEDKAIKNLYASEYIWNSIKDNKTVGIIGEDKEKGLTYVAEPIGVICGVTPTTNPTSTTIFKAMIAIKTGNPIIFAFHPSAQESSKRAAEVVLEAAMKAGAPKDIIQWIEVPSIEATKQLMNHKGIALVLATGGSGMVKSAYSTGKPALGVGPGNVPSYIEKTAHIKRAVNDIIGSKTFDNGMICASEQVVVIDKEIYKDVTNEFKAHQAYFVKKDELQRLENAIMNEQKTGIKPDIVGKSAVEIAELAGIPVPENTKLIIAEISGVGSDYPLSREKLSPVLALVKAQSTKQAFQICEDTLHFGGLGHTAVIHTEDETLQKDFGLRMKACRVLVNTPSAVGGIGDMYNELIPSLTLGCGSYGRNSISHNVSATDLLNIKTIAKRRNNTQIFKVPAQIYFEENAIMSLTTMDKIEKVMIVCDPGMVEFGYTKTVENVLRQRTEQPQIKIFSEVEPNPSTNTVYKGLEMMVDFQPDTIIALGGGSAMDAAKAMWMFFEHPETSFFGAKQKFLDIGKRTYKIGMPENATFICIPTTSGTGSEVTPFAVITDSETNVKYPLADFALTPDVAIIDPQFVMSVPKSVTADTGMDVLTHAMESYVSVMASDYTRGLSLQAIKLTFEYLKSSVEKGDKVSREKMHNASTLAGMAFANAFLGIAHSIAHKIGGEYGIPHGRANAILLPHIIRYNAKDPQKHALFPKYEFFRADTDYADIAKFLGLKGNTTEALVESLAKAVYELGQSVGIEMNLKSQGVSEEELNESIDRMAELAFEDQCTTANPKEALISEIKDIIQTSYDYKQ

SEQ ID NO: 6 - protein, 367 aa, Staphylococcus aureus
  REGION 1..367: note = MISC_FEATURE - E. coli glycerol dehydrogenase (Ec_gldA) amino acid sequence
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 7 - protein, 365 aa, Klebsiella pneumoniae
  REGION 1..365: note = MISC_FEATURE - K. pneumoniae glycerol dehydrogenase (Kpne_gldA) amino acid sequence
MLKVIQSPAKYLQGPDAAVLFGQYAKNLAESFFVIADDFVMKLAGEKVVNGLQSHDIRCHAERFNGECSHAEINRLMAILQKQGCRGVVGIGGGKTLDTAKAIGYYQKLPVVVIPTIASTDAPTSALSVIYTEAGEFEEYLIYPKNPDMVVMDTAIIAKAPVRLLVSGMGDALSTWFEAKACYDARATSMAGGQSTEAALSLARLCYDTLLAEGEKARLAAQAGVVTEALERIIEANTYLSGIGFESSGLAAAHAIHNGFTILEECHHLYHGEKVAFGTLAQLVLQNSPMDEIETVLGFCQRVGLPVTLAQMGVKEGIDAKIAAVAKATCAEGETIHNMPFAVTPESVHAAILTADLLGQQWLAR

SEQ ID NO: 8 - protein, 367 aa, Enterococcus aerogenes
  REGION 1..367: note = MISC_FEATURE - E. aerogenes glycerol dehydrogenase (Eaer_gldA) amino acid sequence
MDRIIQSPGKYIQGAGAIKRLGEYLKPLAERWLIIGDKFVLGFAEEQLRTSLGGAGLVAEIAPFGGECSQNEINRLRDIASSAQCHAVLGIGGGKTLDTAKALAHYMHLPVVVAPTIASTDAPCSALSVIYTDDGEFESYLMLPHNPNMVVVDTQIVAAAPARLLAAGIGDALATWFEARACSRSGATTMAGGKCTQAALALAELCYNTLVEEGEKAMLAAEQHVVTPALERVIEANTYLSGVGFESGGLAAAHAIHNGLTAIPDAHHFYHGEKVAFGTLTQLVLENAPVEEIETAAALCHSVGLPITLAQLDIKGDIPAKMRTVAEAACAEGETIHNMPGGACADQVYAALLVADQYGQRFLQEWE

SEQ ID NO: 9 - protein, 364 aa, Yersinia aldovae
  REGION 1..364: note = MISC_FEATURE - Y. aldovae glycerol dehydrogenase (Eaer_gldA) amino acid sequence
MLKVIQSPSKYIQGANALQSIGEFAKLLANNYFIIADDFVMKLTADTVGTSLQTCELKSHFSRFNGECSRQEIERLTVELKKYGCNGVIGIGGGKTLDTAKAIAHYQHIPVVVVPTIASTDAPTSALSVIYTEQGEFAEYLIYPKNPDIVLMDTTIIAKAPVRLLVAGMGDALSTYFEAQACFDAKAISMAGGASTLAAITLARLCYETLLAEGYKAKLAVEAGVVTEAVERIIEANTYLSGIGFESSGLAAAHAIHNGFTVLEECHHLYHGEKVAFGTLTQLVLQNSSMEEIETVLSFCQQLGLPITLAEMGVTQDLECKIRAVAQASCAEGETIHNMPFKVTADSVYAAIIVADRLGQAFLN

SEQ ID NO: 10 - protein, 584 aa, Saccharomyces cerevisiae
  REGION 1..584: note = MISC_FEATURE - S. cerevisiae dihydroxyacetone kinase (S. cerevisiae DAK1) amino acid sequence
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 11 - protein, 572 aa, Klebsiella pneumoniae
  REGION 1..572: note = MISC_FEATURE - K. pneumoniae dihydroxyacetone kinase (dhaK)
MTTKQFQFDSDPLNSALAATAEASGLAYLPKSKVIYYPLTNDKVTLISGGGAGHEPAQTGFVGPGLLDAAVSGQIFASPSTKQIIAGVNAVKSQRGSIIIVMNYTGDVIHFGMAAEQLRSRYDYHAELVSIGDDISVNKKAGRRGLAGTVLVHKIAGHLARDGWDVGVLAEALRTTAANLATVAASLEHCTVPGRKFETELAADEMEIGMGIHNEPGVKTIKIGKVESLLDELVDKFEPSKQDFVPFNKGDEVVLLVNSLGGVSSLELHAIANIAQTKFEKVLGVKTVRLIVGNFMAAFNGPGFSLTLLNVTTTAKKGNFDVLGALDAPVSTAAWPSLQQKDKPANGGVQEEKETDSDKPAEPTGIKADGKLFKAMIESAVDDLKKEEPQITKYDTIAGDGDCGETLLAGGDGILDAIKNKKIDLDDAAGVADISHIVENSMGGTSGGLYSIFFSGLVVGIKETKAKELSVDVFAKACETALETLSKYTQARVGDRTLMDALVPFVETLSKTKDFAKAVEAARKGADETSKLPANFGRASYVNEEGLENIPDPGALGLAVIFEGLLKAWEKK

SEQ ID NO: 12 - protein, 580 aa, Yarrowia lipolytica
  REGION 1..580: note = MISC_FEATURE - Y. lipolytica dihydroxyacetone kinase (Ylip_DAK1) amino acid sequence
MDKHFINDPEVLVLDGLKSLADMNKTLTVHEEGKFIYFHDYNKKNVSVISGGGAGHEPTHSSFVGKGMLTAAVSGSIFASPSSKQIYTGIKQVESEAGTLVICKNYTGDILHFGMALEKQRTAGKKAELIAVADDVSVGRKKSGKVGRRGLSGTVLVHKIAGAAAARGLPLEAVTTIAKAAIDNLVSIGASLAHVHVPGHEPIAKEDEMKHDEMELGMGIHNEPGCKRISPIPSIDDLIAQMLKQMLDQSDKDRAYVKIEGDDEVVLLMNNLGGLSMLEFSAISHKVKEALAKEYKINPVRIFAGPFTTSLNGLGFGITLLRTTDRVKVEGEEYSLVDLIDQPVEAIGWPLCQPSDLKSKNKIGNVSIEEGQKDVKSPVTVDKEKVRQAIVNSMENLIKAEPKITKFDTMAGDGDCGTTLKRGAEGVLKFVKSDKFSDDPIRIVRDIADVIEDNMDGTSGALYAIFFHGFAKGMKDTLEKSKDISSKTWAAGLKVALDTLFKYTPARPGDSTMCDALVPFVETFVKTNDLNAAVEEARKGADATADMQAKLGRAVYVGDDVKVPDAGALGVVAIVEGFTK

SEQ ID NO: 13 - protein, 580 aa, Schizosaccharomyces pombe; identical to SEQ ID NO: 12
  REGION 1..580: note = MISC_FEATURE - S. pombe dihydroxyacetone kinase (Spom_DAK1) amino acid sequence

SEQ ID NO: 14 - protein, 246 aa, Escherichia coli
  REGION 1..246: note = MISC_FEATURE - E. coli pyruvate-formate lyase maturation enzyme PflA
MSVIGRIHSFESCGTVDGPGIRFITFFQGCLMRCLYCHNRDTWDTHGGKEVTVEDLMKEVVTYRHFMNASGGGVTASGGEAILQAEFVRDWFRACKKEGIHTCLDTNGFVRRYDPVIDELLEVTDLVMLDLKQMNDEIHQNLVGVSNHRTLEFAKYLANKNVKVWIRYVVVPGWSDDDDSAHRLGEFTRDMGNVEKIELLPYHELGKHKWVAMGEEYKLDGVKPPKKETMERVKGILEQYGHKVMF

SEQ ID NO: 15 - protein, 760 aa, Escherichia coli
  REGION 1..760: note = MISC_FEATURE - E. coli pyruvate-formate lyase PflB
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 16 - protein, 291 aa, Danio rerio
  REGION 1..291: note = MISC_FEATURE - D. rerio aquaporin 9 (Drer_T3) amino acid sequence
MEYLENIRNLRGRCVLRRDIIREFLAELLGTFVLILFGCGSVAQTVLSREAKGQLLTIHFGFTLGVMLAVYMAGGVSGGHVNPAVSLAMVVLRKLPLKKFPVYVLAQFLGAFFGSCAVYCLYYDAFTEFANGELAVTGPNVTAGIFASYPREGLSLLNGFIDQVIGAGALVLCILAVVDKKNIGAPKGMEPLLVGLSILAIGVSMALNCGYPINPARDLGPRLFTAIAGWGLTVFSAGNGWWWVPVVGPMVGGVVGAAIYFLMIEMHHPENDKNLEDDNSLKDKYELNTVN

SEQ ID NO: 17 - protein, 592 aa, Zygosaccharomyces rouxii
  REGION 1..592: note = MISC_FEATURE - Z. rouxii ZYRO0E01210p (Zrou_T5) amino acid sequence
MGKRTQGFMDYVFSRTSTAGLKGARLRYTAAAVAVIGFALFGYDQGLMSGLITGDQFNKEFPPTKSNGDNDRYASVIQGAVTACYEIGCFFGSLFVLFFGDAIGRKPLIIFGAIIVIIGTVISTAPFHHAWGLGQFVVGRVITGVGTGFNTSTIPVWQSEMTKPNIRGAMINLDGSVIAFGTMIAYWLDFGFSFINSSVQWRFPVSVQIIFALVLLFGIVRMPESPRWLMAKKRPAEARYVLACLNDLPENDDAILAEMTSLHEAVNRSSNQKSQMKSLFSMGKQQNFSRALIASSTQFFQQFTGCNAAIYYSTVLFQTTVQLDRLLAMILGGVFATVYTLSTLPSFYLVEKVGRRKMFFFGALGQGISFIITFACLVNPTKQNAKGAAVGLYLFIICFGLAILELPWIYPPEIASMRVRAATNAMSTCTNWVTNFAVVMFTPVFIQTSQWGCYLFFAVMNFIYLPVIFFFYPETAGRSLEEIDIIFAKAHVDGTLPWMVAHRLPKLSMTEVEDYSQSLGLHDDENEKEEYDEKEAEANAALFQVETSSKSPSSNRKDDDAPIEHNEVQESNDNSSNSSNVEAPIPVHHNDP

(19) United States
(12) Patent Application Publication
(10) Pub. No.: US 2024/0218401 A1
(43) Pub. Date: Jul. 4, 2024

(54) PROCESS FOR ETHANOL PRODUCTION

(71) Applicant: DANISCO US INC., Palo Alto, CA (US)

(72) Inventors: Paulus Petrus DE WAAL, Echt (NL); Ingrid Maria VUGT-VAN LUTZ, Echt (NL); Jozef Petrus Johannes SCHMITZ, Echt (NL); Hans Marinus Charles Johannes DE BRUIJN, Echt (NL)

(21) Appl. No.: 18/544,037

(22) Filed: Dec. 18, 2023

Related U.S. Application Data
(63) Continuation of application No. 16/649,284, filed on Mar. 20, 2020, now abandoned.

(30) Foreign Application Priority Data
Sep. 26, 2017 (EP) 17193039.9

(51) Int. Cl. C12P 7/10; C07K 14/395; C12N 9/02; C12N 9/04; C12N 9/10

(52) U.S. Cl. CPC C12P 7/10; C07K 14/395; C12N 9/0006; C12N 9/0008; C12N 9/1029; C12Y 101/01006; C12Y 102/0101; C12Y 203/01054; C12P 2203/00

(57) ABSTRACT

The invention relates to a process for the production of ethanol from a composition comprising at least glucose comprising fermenting said composition in the presence of a recombinant yeast; and recovering the ethanol, wherein said yeast comprises one or more genes coding for an enzyme having glycerol dehydrogenase activity, one or more genes coding for an enzyme having dihydroxyacetone kinase activity (E.C. 2.7.1.28 and/or E.C. 2.7.1.29); one or more genes coding for an enzyme in an acetyl-CoA-production pathway and one or more genes coding for an enzyme having at least NAD+ dependent acetylating acetaldehyde dehydrogenase activity (EC 1.2.1.10 or EC 1.1.1.2), and optionally one or more genes coding for a glycerol transporter, wherein the composition comprises an amount of undissociated acetic acid of 10 mM or less. A recombinant yeast having the genes as described above is particularly sensitive towards acetic acid, and the ethanol yield rapidly decreases when the composition contains more than 10 mM undissociated acetic acid.

### CROSS-REFERENCE TO RELATED APPLICATIONS

This application is a continuation of U.S. patent application Ser. No. 16/649,284, filed 20 Mar. 2020, which is the National Stage entry of International Application No. PCT/EP2018/075866, filed 25 Sep. 2018, which claims priority to European Patent Application No. 17193039.9, filed 26 Sep. 2017.

### REFERENCE TO SEQUENCE LISTING

The contents of the electronic submission of the XML file Sequence Listing, named “IFF 32257-US-PCN_SequenceListing.xml” was created on Dec. 15, 2023, and is 32 KB in size, which is hereby incorporated by reference in its entirety.

### FIELD

The invention relates to a process for the production of ethanol from a composition comprising at least glucose.

### BACKGROUND

WO2012/067510 discloses a genetically modified yeast cell comprising exogenous genes coding for pyruvate formate lyase and acetaldehyde dehydrogenase activities as well as glycerol dehydrogenase. This yeast can be used in the production of ethanol. However, the ethanol yield with such yeast is often insufficient. Thus, there is a need for an improved process for the production of ethanol with such cell.

TABLE 1
Description of the sequence listing

| SEQ ID NO: | Description |
|---|---|
| 1 | E. coli bifunctional NAD⁺ dependent acetylating |
| | acetaldehyde/alcohol dehydrogenase (adhE) |
| 2 | E. coli ethanolamine utilizing protein (eutE) |
| 3 | L. plantarum acetaldehyde dehydrogenase (acdH) |
| 4 | L. innocua acetaldehyde dehydrogenase (acdH) |
| 5 | S. aureus acetaldehyde/alcohol dehydrogenase (adhE) |
| 6 | E. coli glycerol dehydrogenase (gldA) |
| 7 | K. pneumoniae glycerol dehydrogenase (gldA) |
| 8 | E. aerogenes glycerol dehydrogenase (gldA) |
| 9 | Y. aldovae glycerol dehydrogenase (gldA) |
| 10 | S. cerevisiae dihydroxyacetone kinase (DAK1) |
| 11 | K. pneumoniae dihydroxyacetone kinase (dhaK) |
| 12 | Y. lipolytica dihydroxyacetone kinase (DAK1) |
| 13 | S. pombe dihydroxyacetone kinase (DAK1) |
| 14 | E. coli pyruvate-formate lyase maturation enzyme PflA |
| 15 | E. coli pyruvate-formate lyase PflB |
| 16 | D. rerio aquaporin 9 (T3) |
| 17 | Z. rouxii ZYRO0E01210p (T5) |

### SUMMARY OF THE INVENTION

The invention provides a process for the production of ethanol from a composition comprising at least glucose comprising fermenting said composition in the presence of a recombinant yeast; and recovering the ethanol, wherein said yeast comprises one or more genes coding for an enzyme having glycerol dehydrogenase activity, one or more genes coding for an enzyme having dihydroxyacetone kinase activity (E.C. 2.7.1.28 and/or E.C. 2.7.1.29); one or more genes coding for an enzyme in an acetyl-CoA-production pathway and one or more genes coding for an enzyme having at least NAD⁺ dependent acetylating acetaldehyde dehydrogenase activity (EC 1.2.1.10 or EC 1.1.1.2), and optionally one or more genes coding for a glycerol transporter, and wherein the composition comprises an amount of undissociated acetic acid of 10 mM or less.

A recombinant yeast having the genes as described above is particularly sensitive towards acetic acid, as compared to non-recombinant yeasts. The ethanol yield rapidly decreases when the composition contains more than 10 mM undissociated acetic acid. The amount of undissociated acetic acid of preferably between 50 μM and 10 mM. The composition may be a lignocellulosic biomass hydrolysate, particularly a corn stover hydrolysate or a corn fiber hydrolysate. Alternatively, the composition may be a starch hydrolysate, such as a corn starch hydrolysate. The enzyme in an acetyl-CoA-production pathway may be an enzyme having pyruvate-formate lyase activity (EC 2.3.1.54) or an enzyme an having an amino acid sequence according to SEQ ID NO: 15 or a functional homologue thereof having a sequence identity of at least 50%. The enzyme having at least NAD⁺ dependent acetylating acetaldehyde dehydrogenase activity may have an amino acid sequence according to SEQ ID NO: 1, 2, 3, 4, or 5 or may be a functional homologue thereof having a sequence identity of at least 50%. The enzyme having at least NAD⁺ dependent acetylating acetaldehyde dehydrogenase activity may catalyse the reversible conversion of acetyl-Coenzyme-A to acetaldehyde and the subsequent reversible conversion of acetaldehyde to ethanol, which enzyme may comprise both NAD⁺ dependent acetylating acetaldehyde dehydrogenase (EC 1.2.1.10 or EC 1.1.1.2) activity and NAD⁺ dependent alcohol dehydrogenase activity (EC 1.1.1.1). The enzyme having glycerol dehydrogenase activity may be a NAD⁺ linked glycerol dehydrogenase (EC 1.1.1.6) or an NADP+ linked glycerol dehydrogenase (EC 1.1.1. 72) or a glycerol dehydrogenase represented by amino acid sequence SEQ ID NO: 6, 7, 8, or 9 a functional homologue thereof a having sequence identity of at least 50%. The yeast may further comprise a deletion or disruption of one or more endogenous genes encoding an enzyme having NAD⁺ dependent formate dehydrogenase (FDH1/2) EC 1.2.1.2. The yeast may further comprise a deletion or disruption of one or more endogenous genes encoding an enzyme having NAD(P)H dependent aldehyde reductase activity (EC 1.2.1.4). The yeast may further comprise a deletion or disruption of one or more endogenous genes encoding a glycerol exporter (e.g. fps1). The yeast may further comprise one or more nucleic acid sequences encoding a heterologous glycerol transporter such as having an amino acid sequence according SEQ ID NO: 16 or 17, or a functional homologue thereof having a sequence identity of at least 50%. The yeast may further comprise a deletion or disruption of one or more endogenous genes encoding a glycerol kinase (EC 2.7.1.30) (e.g. gut1). The yeast may be a yeast which either lacks enzymatic activity needed for NADH-dependent glycerol synthesis or which has reduced enzymatic activity needed for NADH-dependent glycerol synthesis compared to its corresponding wild type (yeast) cell. The yeast may comprise a deletion or disruption of one or more endogenous genes encoding a glycerol-3-phosphate dehydrogenase, which glycerol-3-phosphate dehydrogenase preferably belongs to EC 1.1.5.3, such as gut2, or to EC 1.1.1.8, such as GPD1/2, which cell is preferably 40 free of genes encoding NADH-dependent glycerol 3-phosphate dehydrogenase. The yeast may further comprise a deletion or disruption of one or more endogenous nucleotide sequences encoding a glycerol 3-phosphate phosphohydrolase. The yeast may be selected from Saccharomycesaceae, in particular from the group of Saccharomyces, such as Saccharomyces cerevisiae; Kluyveromyces, such as Kluyveromyces marxianus; Pichia, such as Pichia stipitis or Pichia angusta; Zygosaccharomyces, such as Zygosaccharomyces bailii; and Brettanomyces, such as Brettanomyces intermedius, Issatchenkia, such as Issatchenkia orientalis and Hansenula.

### DETAILED DESCRIPTION

The term “a” or “an” as used herein is defined as “at least one” unless specified otherwise. When referring to a noun (e.g. a compound, an additive, etc.) in the singular, the plural is meant to be included. Thus, when referring to a specific moiety, e.g. “nucleotide”, this means “at least one” of that moiety, e.g. “at least one nucleotide”, unless specified otherwise. The term ‘or’ as used herein is to be understood as ‘and/or’.

The term ‘fermentation’, ‘fermentative’ and the like is used herein in a classical sense, i.e. to indicate that a process is or has been carried out under anaerobic conditions. Anaerobic conditions are herein defined as conditions without any oxygen or in which essentially no oxygen is consumed by the cell, in particular a yeast cell, and usually corresponds to an oxygen consumption of less than 5 mM/h, in particular to an oxygen consumption of less than 2.5 mM/h, or less than 1 mM/h. More preferably O mmol/L/h is consumed (i.e. oxygen consumption is not detectable. This usually corresponds to a dissolved oxygen concentration in the culture broth of less than 5% of air saturation, in particular to a dissolved oxygen concentration of less than 1% of air saturation, or less than 0.2% of air saturation.

The term “yeast” refers to a phylogenetically diverse group of single-celled fungi, most of which are in the division of Ascomycota and Basidiomycota. The budding yeasts (“true yeasts”) are classified in the order Saccharomycesles, with Saccharomyces cerevisiae as the most well-known species.

The term “recombinant” as used herein, refers to a strain containing nucleic acid which is the result of one or more genetic modifications using recombinant DNA technique(s) and/or another mutagenic technique(s). In particular a recombinant cell may comprise nucleic acid not present in a corresponding wild-type cell, which nucleic acid has been introduced into that strain (cell) using recombinant DNA techniques (a transgenic cell), or which nucleic acid not present in said wild-type is the result of one or more mutations—for example using recombinant DNA techniques or another mutagenesis technique such as UV-irradiation—in a nucleic acid sequence present in said wild-type (such as a gene encoding a wild-type polypeptide) or wherein the nucleic acid sequence of a gene has been modified to target the polypeptide product (encoding it) towards another cellular compartment. Further, the term “recombinant (cell)” in particular relates to a strain (cell) from which DNA sequences have been removed using recombinant DNA techniques.

The term “mutated” as used herein regarding proteins or polypeptides means that at least one amino acid in the wild-type or naturally occurring protein or polypeptide sequence has been replaced with a different amino acid, inserted or deleted from the sequence via mutagenesis of nucleic acids encoding these amino acids. Mutagenesis is a well-known method in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989). The term “mutated” as used herein regarding genes means that at least one nucleotide in the nucleic acid sequence of that gene or a regulatory sequence thereof, has been replaced with a different nucleotide, or has been deleted from the sequence via mutagenesis, resulting in the transcription of a protein sequence with a qualitatively of quantitatively altered function or the knock-out of that gene.

In the context of this invention an “altered gene” has the same meaning as a mutated gene.

The term “gene”, as used herein, refers to a nucleic acid sequence containing a template for a nucleic acid polymerase, in eukaryotes, RNA polymerase II. Genes are transcribed into mRNAs that are then translated into protein.

When an enzyme is mentioned with reference to an enzyme class (EC), the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at chem.qmul.ac.uk/iubmb/enzyme. other suitable enzymes that have not (yet) been classified in a specified class but may be classified as such, are meant to be included.

If referred herein to a protein or a nucleic acid sequence, such as a gene, by reference to a accession number, this number in particular is used to refer to a protein or nucleic acid sequence (gene) having a sequence as can be found via ncbi.nlm.nih.gov, (as available on 14 Jun. 2016) unless specified otherwise.

Every nucleic acid sequence herein that encodes a polypeptide also, by reference to the genetic code, describes every possible silent variation of the nucleic acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation.

The term “functional homologue” (or in short “homologue”) of a polypeptide having a specific sequence (e.g. “SEQ ID NO: X”), as used herein, refers to a polypeptide comprising said specific sequence with the proviso that one or more amino acids are substituted, deleted, added, and/or inserted, and which polypeptide has (qualitatively) the same enzymatic functionality for substrate conversion. This functionality may be tested by use of an assay system comprising a recombinant cell comprising an expression vector for the expression of the homologue in yeast, said expression vector comprising a heterologous nucleic acid sequence operably linked to a promoter functional in the yeast and said heterologous nucleic acid sequence encoding the homologous polypeptide of which enzymatic activity for converting acetyl-Coenzyme A to acetaldehyde in the cell is to be tested, and assessing whether said conversion occurs in said cells. Candidate homologues may be identified by using in silica similarity analyses. A detailed example of such an analysis is described in Example 2 of WO2009/013159. The skilled person will be able to derive there from how suitable candidate homologues may be found and, optionally upon codon(pair) optimization, will be able to test the required functionality of such candidate homologues using a suitable assay system as described above. A suitable homologue represents a polypeptide having an amino acid sequence similar to a specific polypeptide of more than 50%, preferably of 60% or more, in particular of at least 70%, more in particular of at least 80%, at least 90%, at least 95%, at least 97%, at least 98% or at least 99% and having the required enzymatic functionality. With respect to nucleic acid sequences, the term functional homologue is meant to include nucleic acid sequences which differ from another nucleic acid sequence due to the degeneracy of the genetic code and encode the same polypeptide sequence.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences.

Amino acid or nucleotide sequences are said to be homologous when exhibiting a certain level of similarity. Two sequences being homologous indicate a common evolutionary origin. Whether two homologous sequences are closely related or more distantly related is indicated by “percent identity” or “percent similarity”, which is high or low respectively. Although disputed, to indicate “percent identity” or “percent similarity”, “level of homology” or “percent homology” are frequently used interchangeably. A comparison of sequences and determination of percent identity between two sequences can be accomplished using a mathematical algorithm. The skilled person will be aware of the fact that several different computer programs are available to align two sequences and determine the homology between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent identity between two amino acid sequences can be determined using the Needleman and Wunsch algorithm for the alignment of two sequences. (Needleman, S. B. and Wunsch, C. D. (1970) J. Mol. Biol. 48, 443-453). The algorithm aligns amino acid sequences as well as nucleotide sequences. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp276-277, emboss.bioinformatics.nl). For protein sequences, EBLOSUM62 is used for the substitution matrix. For nucleotide sequences, EDNAFULL is used. other matrices can be specified. The optional parameters used for alignment of amino acid sequences are a gap-open penalty of 10 and a gap extension penalty of 0.5. The skilled person will appreciate that all these different parameters will yield slightly different results but that the overall percentage identity of two sequences is not significantly altered when using different algorithms.

The homology or identity is the percentage of identical matches between the two full sequences over the total aligned region including any gaps or extensions. The homology or identity between the two aligned sequences is calculated as follows: Number of corresponding positions in the alignment showing an identical amino acid in both sequences divided by the total length of the alignment including the gaps. The identity defined as herein can be obtained from NEEDLE and is labelled in the output of the program as “IDENTITY”.

The homology or identity between the two aligned sequences is calculated as follows: Number of corresponding positions in the alignment showing an identical amino acid in both sequences divided by the total length of the alignment after subtraction of the total number of gaps in the alignment. The identity defined as herein can be obtained from NEEDLE by using the NOBRIEF option and is labelled in the output of the program as “longest-identity”. A variant of a nucleotide or amino acid sequence disclosed herein may also be defined as a nucleotide or amino acid sequence having one or several substitutions, insertions and/or deletions as compared to the nucleotide or amino acid sequence specifically disclosed herein (e.g. in de the sequence listing).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. In an embodiment, conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. In an embodiment, conservative substitutions for each of the naturally occurring amino acids are as follows: Ala to Ser; Arg to Lys; Asn to Gin or His; Asp to Glu; Cys to Ser or Ala; Gin to Asn; Glu to Asp; Gly to Pro; His to Asn or Gin; lie to Leu or Val; Leu to lie or Val; Lys to Arg; Gin or Glu; Met to Leu or lie; Phe to Met, Leu or Tyr; Ser to Thr; Thr to Ser; Trp to Tyr; Tyr to Trp or Phe; and Val to lie or Leu.

Nucleotide sequences of the invention may also be defined by their capability to hybridise with parts of specific nucleotide sequences disclosed herein, respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridization conditions in order to specifically identify sequences varying in identity between 50% and 90%. “Expression” refers to the transcription of a gene into structural RNA (rRNA, tRNA) or messenger RNA (mRNA) with subsequent translation into a protein.

As used herein, “heterologous” in reference to a nucleic acid or protein is a nucleic acid or protein that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

The term “heterologous expression” refers to the expression of heterologous nucleic acids in a host cell. The expression of heterologous proteins in eukaryotic host cell systems such as yeast are well known to those of skill in the art. A polynucleotide comprising a nucleic acid sequence of a gene encoding an enzyme with a specific activity can be expressed in such a eukaryotic system. In some embodiments, transformed/transfected cells may be employed as expression systems for the expression of the enzymes. Expression of heterologous proteins in yeast is well known. Sherman, F., et al., Methods in Yeast Genetics, Cold Spring Harbor Laboratory (1982) is a well-recognized work describing the various methods available to express proteins in yeast. Two widely utilized yeasts are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

As used herein “promoter” is a DNA sequence that directs the transcription of a (structural) gene. Typically, a promoter is located in the 5′-region of a gene, proximal to the transcriptional start site of a (structural) gene. Promoter sequences may be constitutive, inducible or repressible. In an embodiment there is no (external) inducer needed.

By “disruption” is meant (or includes) all nucleic acid modifications such as nucleotide deletions or substitutions, gene knock-outs, (other) which affect the translation or transcription of the corresponding polypeptide and/or which affect the enzymatic (specific) activity, its substrate specificity, and/or or stability. Such modifications may be targeted on the coding sequence or on the pro motor of the gene.

The invention provides a process for the production of ethanol from a composition comprising at least glucose comprising:
fermenting said composition in the presence of a recombinant yeast; and
recovering the ethanol,
   wherein said yeast comprises:
one or more genes coding for an enzyme having glycerol dehydrogenase activity,
one or more genes coding dihydroxyacetone kinase (E.C. 2.7.1.28 and/or E.C. 2.7.1.29);
one or more genes coding for an enzyme in an acetyl-CoA-production pathway and one or more genes coding for an enzyme having at least NAD⁺ dependent acetylating acetaldehyde dehydrogenase activity (EC 1.2.1.10 or EC 1.1.1.2), and optionally
one or more genes coding for a glycerol transporter,
   and wherein the composition comprises an amount of undissociated acetic acid of 10 mM or less.

The inventors have found that a recombinant yeast having the genes as described above is particularly sensitive towards acetic acid, as compared to non-recombinant yeasts. They have surprisingly found that the ethanol yield rapidly decreases when the composition contains more than 10 mM undissociated acetic acid, and that in order to avoid or lessen the negative effect of acetic acid the process should be performed with a composition having an amount of undissociated acetic acid of 10 mM or less, preferably 9 mM or less, 8 mM or less, 7 mM or less, 6 mM or less, 5 mM or less, 4 mM or less, 3 mM or less, 2 mM or less, 1 mM or less.

In an embodiment the composition has an initial undissociated acetic acid of 10 mM or less. In another embodiment, the amount of undissociated acetic acid is 10 mM or less throughout the process.

The lower amount of undissociated acetic acid is less important. In one embodiment, the composition is free of undissociated acetic acid.

In an embodiment, the lower limit of the amount of undissociated acetic acid is 50 μM or more, 55 μM or more, 60 μM or more, 70 μM or more, 80 μM or more, 90 μM or more, 100 μM or more. The recombinant yeast used in the process of the invention comprises a gene encoding an acetylating acetaldehyde dehydrogenase, which allows the yeast to convert acetic acid, which may be present in both lignocellulosic hydrolysates and in corn starch hydrolysates, to ethanol. Although the recombinant yeast used in the process of the invention should in principle be able to consume acetic acid, the inventors have surprisingly found that there is often a residual amount of acetic acid in the fermentation media which remains unconverted. This residual amount of acetic acid may be as large as several millimolar. The inventors found that yeast requires a minimum concentration of undissociated acetic acid of at least 50 μM. Below this concentration, the consumption of acetic acid decreases, even if there is a considerable amount of dissociated acetic acid present in the fermentation media.

The skilled person appreciates that the amount of undissociated acetic acid depends inter Alia on the total amount of acetic acid in the composition (protonated and dissociated) as well o the pH.

In one embodiment the amount of undissociated acetic acid is maintained at a value of at 10 mM by adjusting the pH, e.g. by adding a base.

The process may comprise the step of monitoring the pH. The pH of the composition is preferably kept between 4.2 and 5.2, preferably between 4.5 and 5.0. The lower pH is preferably such that the amount of undissociated acetic acid is 10 mM or less, which inter alia depends on the total amount of acetic acid in the composition.

The skilled person knows how to provide or select a composition having an amount of undissociated acetic acid 10 mM or less. For example, he/she may measure the amount of undissociated acetic acid in a composition and select only those compositions which have an amount of undissociated acetic acid of 10 mM or less.

Alternatively, if the amount of undissociated acetic acid in a composition exceeds 10 mM, the process may comprise, prior to the fermentation step, adding a base (such as NaOH or KOH) until the amount of undissociated acetic acid in a composition has reached a value of 10 mM or less.

The amount of undissociated acetic acid may be analysed by HPLC. HPLC generally measures all acetic acid (i.e. both undissociated, i.e. protonated form and dissociated form of acetic acid) because the mobile phase is typically acidified. In order to measure the amount of undissociated acetic acid in the composition, a suitable approach is to measure the (total) amount of acetic acid of the composition as-is, measure the pH of the composition, and calculate the amount of undissociated acetic acid using the pKa of acetic acid.

In an embodiment the composition is a biomass hydrolysate. Such biomass hydrolysate may be a lignocellulosic biomass hydrolysate. Lignocellulose herein includes hemicellulose and hemicellulose parts of biomass. Also lignocellulose includes lignocellulosic fractions of biomass. Suitable lignocellulosic materials may be found in the following list: orchard primings, chaparral, mill waste, urban wood waste, municipal waste, logging waste, forest thinnings, short-rotation woody crops, industrial waste, wheat straw, oat straw, rice straw, barley straw, rye straw, flax straw, soy hulls, rice hulls, rice straw, corn gluten feed, oat hulls, sugar cane, corn stover, corn stalks, corn cobs, corn husks, switch grass, miscanthus, sweet sorghum, canola stems, soybean stems, prairie grass, gamagrass, foxtail; sugar beet pulp, citrus fruit pulp, seed hulls, cellulosic animal wastes, lawn clippings, cotton, seaweed, trees, softwood, hardwood, poplar, pine, shrubs, grasses, wheat, wheat straw, sugar cane bagasse, corn, corn husks, corn hobs, corn kernel, fiber from kernels, products and by-products from wet or dry milling of grains, municipal solid waste, waste paper, yard waste, herbaceous material, agricultural residues, forestry residues, municipal solid waste, waste paper, pulp, paper mill residues, branches, bushes, canes, corn, corn husks, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, oat hulls, hard or soft wood, organic waste material generated from an agricultural process, forestry wood waste, or a combination of any two or more thereof. Lignocellulose, which may be considered as a potential renewable feedstock, generally comprises the polysaccharides cellulose (glucans) and hemicelluloses (xylans, heteroxylans and xyloglucans). In addition, some hemicellulose may be present as glucomannans, for example in wood-derived feedstocks. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucuronic acid and other hexoses and pentoses occurs under the action of different enzymes acting in concert. In addition, pectins and other pectic substances such as arabinans may make up considerably proportion of the dry mass of typically cell walls from non-woody plant tissues (about a quarter to half of dry mass may be pectins). Lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes.

A preferred composition is a pre-treated cornstover hydrolysate. Another preferred composition is a corn fiber hydrolysate, which is optionally pre-treated.

In another embodiment the composition is a starch hydrolysate, such as a corn starch hydrolysate.

In the context of the invention a “hydrolysate” means a polysaccharide that has been depolymerized through the addition of water to form mono and oligosaccharide sugars. Hydrolysates may be produced by enzymatic or acid hydrolysis of the polysaccharide-containing material.

The recombinant cell comprises one or more genes coding for an enzyme in an acetyl-CoA-production pathway. In an embodiment, the one or more genes coding for an enzyme in an acetyl-CoA-production pathway comprises one or more genes coding for an enzyme having pyruvate-formate lyase activity (EC 2.3.1.54).

The E. coli pyruvate formate lyase is a dimer of Pf1B (encoded by pf1B), whose maturation requires the activating enzyme Pf1AE (encoded by pf1A), radical S-adenosylmethionine, and a single electron donor, which in the case of E. coli is flavodoxin (Buis and Broderick, 2005, Arch. Biochem. Biophys. 433:288-296; Sawers and Watson, 1998, Mol. Microbial. 29:945-954).

A pyruvate formate lyase may have an amino acid sequence according to SEQ ID NO: or may be a functional homologue thereof having a sequence identity of at least 50%, preferably at least 60%, 70%, 75%, 80%, 85%, 90%, 95%, or at least 99%. As herein, a pyruvate-formate lyase catalyses at least the following reaction (I):

$$pyruvate + coenzyme A \text{<->} formate + acetyl coenzyme A \qquad ( I )$$

Suitable nucleic acid sequences coding for an enzyme having pyruvate-formate lyase may in be found in Bifidobacteria, Escherichia, Thermoanaerobacter, Clostridia, Streptococcus, Lactobacillus, Chlamydomonas, Piromyces, Neocallimastix, or Bacillus, in particular in Bacillus licheniformis, Streptococcus thermophilus, Lactobacillus plantarum, Lactobacillus casei, Bifidobacterium adolescentis, Clostridium cel/ulolyticum, Escherichia coli, Chlamydomonas reinharlii Pf1A, Piromyces sp. E2, Neocallimastix frontalis, or in Bifidobacterium adolescentis.

The yeast may also comprise one or more genes coding for an enzyme according to SEQ ID NO: 14 or a functional homologue thereof having a sequence identity of at least 50%, preferably at least 60%, 70%, 75%, 80%, 85%, 90%, 95%, or at least 99%.

The enzyme acetylating acetaldehyde dehydrogenase (EC1.2.1.10 or EC1.1.1.2) catalyses the conversion of acetyl-Coenzyme A to acetaldehyde. This conversion can be represented by the equilibrium reaction formula (II):

acetyl-Coenzyme A+NADH+H+<->acetaldehyde+NAD⁺+Coenzyme A (II)

It is understood that the recombinant yeast used in the process of the invention naturally comprises at least one endogenous gene encoding an acetyl COA synthetase and at least one endogenous gene encoding an alcohol dehydrogenase.

The enzyme having acetylating acetaldehyde dehydrogenase activity is preferably NAD⁺ dependent and may have an amino acid sequence according to SEQ ID NO: 1, 2, 3, 4, or 5 or may be a functional homologue thereof having a sequence identity of at least 50%, preferably at least 60%, 70%, 75%, 80%. 85%, 90% or 95%. The acetylating acetaldehyde may comprise both NAD⁺ dependent acetylating acetaldehyde dehydrogenase (EC 1.2.1.10 or EC 1.1.1.2) activity and NAD⁺ dependent alcohol dehydrogenase activity (EC 1.1.1.1). The nucleic acid sequence encoding the NAD⁺ dependent acetylating acetaldehyde dehydrogenase may in principle originate from any organism comprising a nucleic acid sequence encoding said dehydrogenase. Known acetylating acetaldehyde dehydrogenases that can catalyse the NADH-dependent reduction of acetyl-Coenzyme A to acetaldehyde may in general be divided in three types of NAD⁺ dependent acetylating acetaldehyde dehydrogenase functional homologues:

1) Bifunctional proteins that catalyse the reversible conversion of acetyl-CoA to acetaldehyde, and the subsequent reversible conversion of acetaldehyde to ethanol. An example of this type of proteins is the AdhE protein in E. coli (Gen Bank No: NP_415757). AdhE appears to be the evolutionary product of a gene fusion. The NH2− terminal region of the AdhE protein is highly homologous to aldehyde: NAD⁺ oxidoreductases, whereas the COOH-terminal region is homologous to a family of Fe2+ dependent ethanol: NAD⁺ oxidoreductases (Membrillo-Hernandez et al., (2000) J. Biol. Chem. 275: 33869-33875). The E. coli AdhE is subject to metal-catalyzed 10 oxidation and therefore oxygen-sensitive (Tamarit et al. (1998) J. Biol. Chem. 273:3027-32).

2) Proteins that catalyse the reversible conversion of acetyl-Coenzyme A to acetaldehyde in strictly or facultative anaerobic micro-organisms but do not possess alcohol dehydrogenase activity. An example of this type of proteins has been reported in Clostridium kluyveri (Smith et al. (1980) Arch. Biochem. Biophys. 203: 663-675). An acetylating acetaldehyde dehydrogenase has been annotated in the genome of Clostridium kluyveri DSM 555 (GenBank No: EDK33116). A homologous protein AcdH is identified in the genome of Lactobacillus plantarum (GenBank No: NP_784141). Another example of this type of proteins is the said gene product in Clostridium beijerinckii NRRL 8593 (Toth et al. (1999) Appl. Environ. Microbial. 65: 4973-4980, GenBank No: AAD31841).

3) Proteins that are part of a bifunctional aldolase-dehydrogenase complex involved in 4-hydroxy-2-ketovalerate catabolismuch bifunctional enzymes catalyze the final two steps of the meta-cleavage pathway for catechol, an intermediate in many bacterial species in the degradation of phenols, toluates, naphthalene, biphenyls and other aromatic compounds (Pawlowski and Shingler (1994) Biodegradation 5, 219-236). 4-Hydroxy-2-ketovalerate is first converted by 4-hydroxy-2-ketovalerate aldolase to pyruvate and acetaldehyde, subsequently acetaldehyde is converted by acetylating acetaldehyde dehydrogenase to acetyl-CoA. An example of this type of acetylating acetaldehyde dehydrogenase is the DmpF protein in Pseudomonas sp CF600 (GenBank No: CAA43226) (Shingler et al. (1992) J. Bacterial. 174:711-24). The E. coli MphF protein (Ferrandez et al. (1997) J. Bacterial. 179: 2573-2581, GenBank No: NP_414885) is homologous to the DmpF protein in Pseudomonas sp. CF600.

A suitable nucleic acid sequence may in particular be found in an organism selected from the group of Escherichia, in particular E. coli; Mycobacterium, in particular Mycobacterium marinum, Mycobacterium ulcerans, Mycobacterium tuberculosis; Carboxydothermus, in particular Carboxydothermus hydrogenoformans; Entamoeba, in particular Entamoeba histolytica; Shigel/a, in particular Shigel/a sonnei; Burkholderia, in particular Burkholderia pseudo mallei, Klebsiella, in particular Klebsiella pneumoniae; Azotobacter, in particular Azotobacter vinelandii; Azoarcus sp; Cupriavidus, in particular Cupriavidus taiwanensis; Pseudomonas, in particular Pseudomonas sp. CF600; Pelomaculum, in particular Pelotomaculum thermopropionicum. Preferably, the nucleic acid sequence encoding the NAD⁺ dependent acetylating acetaldehyde dehydrogenase originates from Escherichia, more preferably from E. coli.

Particularly suitable is an mhpF gene from E. coli, or a functional homologue thereof. This gene is described in Fernindez et al. (1997) J. Bacterial. 179:2573-2581. Good results have been obtained with S. cerevisiae, wherein an mhpF gene from E. coli has been incorporated. In a further advantageous embodiment the nucleic acid sequence encoding an (acetylating) acetaldehyde dehydrogenase is from Pseudomonas, in particular dmpF, e.g. from Pseudomonas sp. CF600.

The acetylating acetaldehyde dehydrogenase may be a wild type enzyme. Further, an acetylating acetaldehyde dehydrogenase (or nucleic acid sequence encoding such activity) may for instance be selected from the group of Escherichia coli adhE, Entamoeba histolytica adh2, Staphylococcus aureus adhE, Piromyces sp.E2 adhE, Clostridium kluyveri EDK33116, Lactobacillus plantarum acdH, Escherichia coli eutE, Listeria innocua acdH, and Pseudomonas putida YP 001268189. For sequences of some of these enzymes, nucleic acid sequences encoding these enzymes and methodology to incorporate the nucleic acid sequence into a host cell, reference is made to WO2009/013159, in particular Example 3, Table 1 (page 26) and the Sequence ID numbers mentioned therein, of which publication Table 1 and the sequences represented by the Sequence ID numbers mentioned in said Table are incorporated herein by reference.

The enzyme glycerol dehydrogenase catalyzes at least the following reaction (III):

glycerol+NAD+<->glycerone+NADH+H+ (III)

Thus, the two substrates of this enzyme are glycerol and NAD⁺, whereas its three products are glycerone, NADH, and H+. Glycerone and dihydroxyacetone are herein synonyms.

This enzyme belongs to the family of oxidoreductases, specifically those acting on the CHOH group of donor with NAD⁺ or NADP+ as acceptor. The systematic name of this enzyme class is glycerol: NAD⁺2-oxidoreductase. other names in common use include glycerin dehydrogenase, and NAD⁺-linked glycerol dehydrogenase. This enzyme participates in glycerolipid metabolism.

Structural studies have shown that the enzyme is zinc-dependent with the active site lying between the two domains of the protein.

In an embodiment the enzyme having glycerol dehydrogenase activity is preferably a NAD⁺ linked glycerol dehydrogenase (EC 1.1.1.6). Such enzyme may be from bacterial origin or for instance from fungal origin. An example is gldA from E. coli.

The enzyme having glycerol dehydrogenase activity may also be a NADP+ linked glycerol dehydrogenase (EC 1.1.1.72).

When the recombinant yeast is used for ethanol production, which typically takes place under anaerobic conditions, NAD⁺ linked glycerol dehydrogenases are preferred.

In an embodiment the recombinant yeast comprises one or more genes encoding a heterologous glycerol dehydrogenase represented by amino acid sequence SEQ ID NO: 6, 7, 8, or 9 a functional homologue thereof a having sequence identity of at least 50%, preferably at least 60%, 70%, 75%, 80%. 85%, 90% or 95%.

It is understood that the recombinant yeast comprises a nucleic acid coding for an enzyme having dihydroxyacetone kinase activity. The enzyme dihydroxyacetone kinase catalyzes at least one of the following reactions:

$$EC 2.7 \text{.1} \text{.28} : ATP + D - glyceraldehyde \text{<=>} ADP + D - glyceraldhyde 3 - phosphate \qquad ( IV )$$

$$or$$

$$EC 2.7 \text{.1} \text{.29} : ATP + glycerone \text{<=>} ADP + glycerone phosphate \qquad ( V )$$

This family consists of examples of the single chain form of dihydroxyacetone kinase (also called glycerone kinase) that uses ATP (EC 2.7.1.29 or EC 2.7.1.28) as the phosphate donor, rather than a phosphoprotein as in Escherichia coli. This form has separable domains homologous to the K and L subunits of the E. coli enzyme, and is found in yeasts and other eukaryotes and in some bacteria, including Citrobacter freundii. The member from tomato has been shown to phosphorylate dihydroxyacetone, 3,4-dihydroxy-2-butanone, and some other aldoses and ketoses. Members from mammals have been shown to catalyse both the phosphorylation of dihydroxyacetone and the splitting of ribonucleoside diphosphate-X compounds among which FAD is the best substrate. In yeast there are two isozymes of dihydroxyacetone kinase (Dak1 and Dak2). In an embodiment the recombinant yeast comprises endogenous OAK which is overexpressed.

The enzyme having dihydroxy acetone kinase activity may be encoded by an endogenous gene, e.g. a DAK1, which endogenous gene is preferably placed under control of a constitutive promoter. The recombinant cell may comprise a genetic modification that increases the specific activity of dihydroxyacetone kinase in the cell.

In an embodiment the recombinant yeast comprises one or more nucleic acid sequences encoding a dihydroxy acetone kinase represented by amino acid sequence according to SEQ ID NO: 10, 11, 12, or 13 or by a functional homologue thereof having a sequence identity of at least 50%, preferably at least 60%, 70%, 75%, 80%. 85%, 90% or 95%, which gene is preferably placed under control of a constitutive promoter.

In an embodiment the recombinant cell comprises a deletion or disruption of one or more endogenous genes encoding an enzyme having NAD⁺ dependent formate dehydrogenase (FDH1 or FDH2, (EC1.2.1.2). As used herein, an NAD⁺ dependent formate dehydrogenase catalyses at least the oxidation of formate to bicarbonate, donating the electrons to NAD⁺. In the recombinant cell, the specific formate dehydrogenase activity is preferably reduced by at least a factor 0.8, 0.5, 0.3, 0.1, 0.05 or 0.01 as compared to a strain which is genetically identical except for the genetic modification causing the reduction in specific activity, preferably under anaerobic conditions. Formate dehydrogenase activity may be determined as described by Overkamp et al. (2002, Yeast 192509-520). Preferably, formate dehydrogenase activity is reduced in the host cell by one or more genetic modifications that reduce the expression of or inactivates a gene encoding an formate dehydrogenase. Preferably, the genetic modifications reduce or inactivate the expression 40 of each endogenous copy of the gene encoding a specific formate dehydrogenase in the cell's genome. A given cell may comprise multiple copies of the gene encoding a specific formate dehydrogenase with one and the same amino acid sequence as a result of di-, poly- or aneuploidy. In such instances preferably the expression of each copy of the specific gene that encodes the formate dehydrogenase is reduced or inactivated. Alternatively, a cell may contain several different (iso)enzymes with formate dehydrogenase activity that differ in amino acid sequence and that are each encoded by a different gene. In such instances, in some embodiments of the invention it may be preferred that only certain types of the isoenzymes are reduced or inactivated while other types remain unaffected. Preferably, however, expression of all copies of genes encoding (iso)enzymes with formate dehydrogenase activity is reduced or inactivated.

A gene encoding formate dehydrogenase activity may be inactivated by deletion of at least part of the gene or by disruption of the gene, whereby in this context the term gene also includes any non-coding sequence up- or down-stream of the coding sequence, the (partial) deletion or inactivation of which results in a reduction of expression of formate dehydrogenase activity in the host cell. A preferred gene encoding a formate dehydrogenase whose activity is to be reduced or inactivated in the cell of the invention is the S. cerevisiae FOHi as described by van den Berg and Steensma (1997, Yeast 13:551-559). In some strains of S. cerevisiae a second gene encoding a formate dehydrogenase is active, i.e. the FDH2, see e.g. Overkamp et al. (2002, supra). Another preferred gene encoding a formate dehydrogenase whose activity is to be reduced or inactivated in the cell of the invention therefore is an S. cerevisiae FDH2 as described by Overkamp et al. (2002, supra).

In an embodiment the recombinant cell comprises a deletion or disruption of one or more endogenous genes encoding an enzyme having NAD(P)H dependent aldehyde reductase activity (EC 1.2.1.4). As used herein, an aldehyde reductase catalyzes at least the following reaction:

$$acetaldehyde + NAD ( P ) + \text{<->} acetic acid + NAD ( P ) H \qquad ( VI )$$

In an embodiment the recombinant cell comprises a deletion or disruption of one or more endogenous nucleotide sequences encoding a glycerol exporter (e.g. FPS1).

In an embodiment the recombinant cell comprises one or more genes coding for a glycerol transporter or an enzyme an having an amino acid sequence according to SEQ ID NO: 16 or SEQ ID NO: 17 or a functional homologue thereof having a sequence identity of at least 50%, preferably at least 60%, 70%, 75%, 80%. 85%, 90%, 95%, or at least 99%. Any glycerol that is externally available in the medium (e.g. from the backset in corn mash) or secreted after internal cellular synthesis may be transported into the cell and converted to ethanol.

In another embodiment the recombinant cell comprises a deletion or disruption of one or more endogenous nucleotide sequences encoding a glycerol kinase (EC 2.7.1.30). An example of such an enzyme is Gut1. As used herein, a glycerol kinase catalyzes at least the following reaction:

$$glycerol + phosphate 7 glycerol phosphate \qquad ( VII )$$

In an embodiment the recombinant yeast either lacks enzymatic activity needed for NADH-dependent glycerol synthesis or the yeast has reduced enzymatic activity needed for NADH-dependent glycerol synthesis compared to its corresponding wild type (yeast).

In one embodiment the recombinant yeast comprises a deletion or disruption of one or more endogenous nucleotide sequences encoding a glycerol-3-phosphate dehydrogenase. Such a deletion or disruption may result in decrease or removal of enzymatic activity. As used herein, a glycerol 3-phosphate dehydrogenase catalyzes at least the following reaction:

$$dihydroxyacetone phosphate + NADH 7 glycerol phosphate + N A D + \qquad ( VIII )$$

Glycerol-3-phosphate dehydrogenase may be entirely deleted, or at least a part is deleted which encodes a part of the enzyme that is essential for its activity. In particular, good results have been achieved with a S. cerevisiae cell, wherein the open reading frames of the GPD1 gene and of the GPD2 gene have been inactivated. Inactivation of a structural gene (target gene) can be accomplished by a person skilled in the art by synthetically synthesizing or otherwise constructing a DNA fragment consisting of a selectable marker gene flanked by DNA sequences that are identical to sequences that flank the region of the host cell's genome that is to be deleted. In particular, good results have been obtained with the inactivation of the GPD1 and GPD2 genes in Saccharomyces cerevisiae by integration of the marker genes kanMX and hphMX4. Subsequently this DNA fragment is transformed into a host cell. Transformed cells that express the dominant marker gene are checked for correct replacement of the region that was designed to be deleted, for example by a diagnostic polymerase chain reaction or Southern hybridization. The deleted or disrupted glycerol-3-phosphate dehydrogenase preferably belongs to EC 1.1.5.3, such as GUT2, or to EC 1.1.1.8, such as GPD1 and or GPD2. In embodiment the cell is free of genes encoding NADH-dependent glycerol-3-phosphate dehydrogenase. Both GPD1 and GPD2 genes may be deleted or disrupted, although it is preferred that GPD2, but not GPD1 is deleted or disrupted. WO2011/010923 describes methods to delete or disrupt a glycerol-3-phosphate dehydrogenase.

In another embodiment the recombinant yeast comprises a deletion or disruption of one or more endogenous nucleotide sequences encoding a glycerol 3-phosphate phosphohydrolase, such as S. cerevisiae GPP1 or GPP2. Such a deletion or disruption may result in decrease or removal of enzymatic activity.

The recombinant cell according to the invention may be subjected to evolutionary engineering to improve its properties. Evolutionary engineering processes are known processes. Evolutionary engineering is a process wherein industrially relevant phenotypes of a microorganism, herein the recombinant cell, can be coupled to the specific growth rate and/or the affinity for a nutrient, by a process of rationally set-up natural selection. Evolutionary Engineering is for instance described in detail in Kuijper, M, et al, FEMS, Eukaryotic cell Research 5(2005) 925-934, WO2008041840 and WO20091124 72. After the evolutionary engineering the resulting pentose fermenting recombinant cell is isolated. The isolation may be executed in any known manner, e.g. by separation of cells from a recombinant cell broth used in the evolutionary engineering, for instance by taking a cell sample or by filtration or centrifugation.

In an embodiment, the recombinant cell is marker-free. As used herein, the term “marker” refers to a gene encoding a trait or a phenotype which permits the selection of, or the screening for, a host cell containing the marker. Marker-free means that markers are essentially absent in the recombinant cell. Being marker-free is particularly advantageous when antibiotic markers have been used in construction of the recombinant cell and are removed thereafter. Removal of markers may be done using any suitable prior art technique, e.g. intramolecular recombination.

In one embodiment, the recombinant cell is constructed on the basis of an inhibitor tolerant host cell, wherein the construction is conducted as described hereinafter. Inhibitor tolerant host cells may be selected by screening strains for growth on inhibitors containing materials, such as illustrated in Kadar et al, Appl. Biochem. Biotechnol. (2007), Vol. 136-140, 847-858, wherein an inhibitor tolerant S. cerevisiae strain ATCC 26602 was selected.

To increase the likelihood that enzyme activity is expressed at sufficient levels and in active form in the recombinant cell, the nucleotide sequence encoding these enzymes, as well as the Rubisco enzyme and other enzymes of the disclosure are preferably adapted to optimise their codon usage to that of the cell in question.

The adaptiveness of a nucleotide sequence encoding an enzyme to the codon usage of a cell may be expressed as codon adaptation index (CAI). The codon adaptation index is herein defined as a measurement of the relative adaptiveness of the codon usage of a gene towards the codon usage of highly expressed genes in a particular cell or organism. The relative adaptiveness (w) of each codon is the ratio of the usage of each codon, to that of the most abundant codon for the same amino acid. The CAI index is defined as the geometric mean of these relative 25 adaptiveness values. Non-synonymous codons and termination codons (dependent on genetic code) are excluded. CAI values range from Oto 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence preferably has a CAI of at least 0.2, 0.3, 0.4, 0.5, 0.6, 0.7, 0.8 or 0.9. Most preferred are the sequences which have been codon optimised for expression in the host cell in question such as e.g. S. cerevisiae cells.

The recombinant yeast may be selected from Saccharomycesaceae, in particular from the group of Saccharomyces, such as Saccharomyces cerevisiae; Kluyveromyces, such as Kluyveromyces marxianus; Pichia, such as Pichia stipitis or Pichia angusta; Zygosaccharomyces, such as Zygosaccharomyces bailii; and Brettanomyces, such as Brettanomyces intermedius, Issatchenkia, such as/ssatchenkia orientalis and Hansenula.

1. A process for the production of ethanol from a composition comprising at least glucose comprising:
fermenting said composition in the presence of a recombinant yeast; and
recovering the ethanol,
wherein said yeast comprises:
one or more genes coding for an enzyme having glycerol dehydrogenase activity,
one or more genes coding for an enzyme having dihydroxyacetone kinase activity (E.C. 2.7.1.28 and/or E.C. 2.7.1.29);
one or more genes coding for an enzyme in an acetyl-CoA-production pathway and
one or more genes coding for an enzyme having at least NAD⁺ dependent acetylating acetaldehyde dehydrogenase activity (EC 1.2.1.10 or EC 1.1.1.2), and one or more genes coding for a glycerol transporter, and wherein the composition comprises an amount of undissociated acetic acid of 10 mM or less.

2. Process according to claim 1 wherein the composition comprises an amount of undissociated acetic acid of between 50 μM and 10 mM.

3. (canceled)

4. Process according to claim 1, wherein the composition is a starch hydrolysate.

5. Process according to claim 1, wherein the enzyme in an acetyl-CoA-production pathway is an enzyme having pyruvate-formate lyase activity (EC 2.3.1.54) or an enzyme having an amino acid sequence according to SEQ ID NO: 15 or a functional homologue thereof having a sequence identity of at least 50%.

6. Process according to claim 1, wherein the enzyme having at least NAD⁺ dependent acetylating acetaldehyde dehydrogenase activity has an amino acid sequence according to SEQ ID NO: 1, 2, 3, 4, or 5 which is a functional homologue thereof having a sequence identity of at least 50%.

7. Process according to claim 1, wherein the enzyme having at least NAD⁺ dependent acetylating acetaldehyde dehydrogenase activity catalyses the reversible conversion of acetyl-Coenzyme-A to acetaldehyde and the subsequent reversible conversion of acetaldehyde to ethanol.

8. Process according to claim 1, wherein the enzyme having glycerol dehydrogenase activity is a NAD⁺ linked glycerol dehydrogenase (EC 1.1.1.6) or an NADP⁺ linked glycerol dehydrogenase (EC 1.1.1.72) or a glycerol dehydrogenase represented by amino acid sequence SEQ ID NO: 6, 7, 8, or 9 a functional homologue thereof a having sequence identity of at least 50%.

9. Process according to claim 1, wherein the yeast comprises a deletion or disruption of one or more endogenous genes encoding an enzyme having NAD⁺ dependent formate dehydrogenase (FDH1/2) EC 1.2.1.2.

10. Process according to claim 1, wherein the yeast comprises a deletion or disruption of one or more endogenous genes encoding an enzyme having NAD(P)H dependent aldehyde reductase activity (EC 1.2.1.4).

11. Process according to claim 1, wherein the yeast comprises a deletion or disruption of one or more endogenous genes encoding a glycerol exporter.

12. Process according to claim 1, wherein the yeast comprises one or more nucleic acid sequences encoding a heterologous glycerol transporter such as having an amino acid sequence according SEQ ID NO: 16 or 17, or a functional homologue thereof having a sequence identity of at least 50%.

13. Process according to claim 1, wherein the yeast comprises a deletion or disruption of one or more endogenous genes encoding a glycerol kinase (EC 2.7.1.30).

14. Process according to claim 1, wherein the yeast either lacks enzymatic activity needed for NADH-dependent glycerol synthesis or wherein said cell has reduced enzymatic activity needed for NADH-dependent glycerol synthesis compared to its corresponding wild type yeast cell.

15. Process according to claim 1, wherein the yeast comprises a deletion or disruption of one or more endogenous genes encoding a glycerol-3-phosphate dehydrogenase.

16. Process according to claim 1, wherein the yeast comprises a deletion or disruption of one or more endogenous nucleotide sequences encoding a glycerol 3-phosphate phosphohydrolase.

17. Process according to claim 1, wherein the yeast is a Saccharomyces cerevisiae a Kluyveromyces marxianus, a Pichia stipites, a Pichia angusta, a Zygosaccharomyces bailii, a Brettanomyces intermedius, a lssatchenkia orientalis, or a Hansenula.
